(19) United States
(12) Patent Application Publication
(10) Pub. No.: US 2008/0188926 A1
(43) Pub. Date: Aug. 7, 2008

(54) Stem Cell Coated Stent

(75) Inventor: Susan Rea, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/670,227

(22) Filed: Feb. 1, 2007

(51) Int. Cl. A61F 2/82; A61F 2/84

(52) U.S. Cl. 623/1.42; 623/1.46

(57) ABSTRACT

A method of treating a vascular condition includes applying a plurality of stem cells to an exterior surface of a stent, and enveloping the applied stem cells with a topcoat layer. In addition, the method includes delivering the stent with applied stem cells and topcoat to a treatment region of a vessel within a body; and applying an electrical field to the stent for a predetermined time. A system for treating a vascular condition includes a catheter, a stent disposed on the catheter, at least one layer of stem cells disposed on an exterior surface of the stent, and a topcoat layer surrounding the layer of stem cells. In addition, the system includes at least one electrical lead attached to the stent, the electrical lead operable to induce an electrical field around the stent.

### TECHNICAL FIELD

This invention relates generally to implantable devices that are used for treating vascular conditions and coatings for the devices.

### BACKGROUND OF THE INVENTION

Stents have become popular medical devices for treatment of vascular conditions. One difficulty with such devices is increasing the biocompatibility of the stent. Previously, this problem has been addressed by incorporating pharmaceutical ingredients and stent shape.

One attempt to help increase biocompatibility includes the use of a radial groove to encourage ingrowth of smooth muscle cells. However, such attempts do not address problems caused by tissues closer to the stent surface, such as the endothelial lining of vessel walls.

In an intact artery, the intima consists mainly of endothelial cells oriented longitudinally to provide good mechanical support and proper biologic function. When the vessel is injured and partially or completely denuded during stent deployment, the new endothelial layer that forms upon healing has a disordered, patchwork appearance that may undesirably affect both the mechanical and biological response capabilities of the vessel.

In addition to attaching drugs to the stent surface, other treatment regimens have been explored. For example, the application of a low electrical field is known to promote angiogenesis and have potentially desired results. For example, application of a field can assist in uptake regulation of nitric oxide (NO) and vascular endothelial growth factor (VEGF) proteins

It would be desirable, therefore, to provide a method of treating a vascular condition that would overcome the limitations and disadvantages inherent in the devices described above.

### SUMMARY OF THE INVENTION

A first aspect of the invention provides a method of treating a vascular condition. The method includes applying a plurality of stem cells to an exterior surface of a stent, and enveloping the applied stem cells with a topcoat layer. In addition, the method includes delivering the stent with applied stem cells and topcoat to a treatment region of a vessel within a body; and applying an electrical field to the stent for a predetermined time.

Another aspect of the invention provides a system for treating a vascular condition. The system includes a catheter, a stent disposed on the catheter, at least one layer of stem cells disposed on an exterior surface of the stent, and a topcoat layer surrounding the layer of stem cells. In addition, the system includes at least one electrical lead attached to the stent, the electrical lead operable to induce an electrical field around the stent.

Another aspect of the invention provides a system for treating a vascular condition. The system includes a catheter, a stent disposed on the catheter, at least one layer of stem cells disposed on an exterior surface of the stent, and a topcoat layer surrounding the layer of stem cells. In addition, the system includes at least one magnetic device operable to induce an electrical field around the stent

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is an illustration of a system for treating a vascular condition including a stent coupled to a catheter, in accordance with one embodiment of the current invention;

FIG. 2 is a cross-sectional perspective view of a stent with an electrical lead, in accordance with one embodiment of the current invention; and

FIG. 3 is a flow diagram of a method treating a vascular condition, in accordance with one embodiment of the current invention.

### DETAILED DESCRIPTION

The invention will now be described by reference to the drawings wherein like numbers refer to like structures.

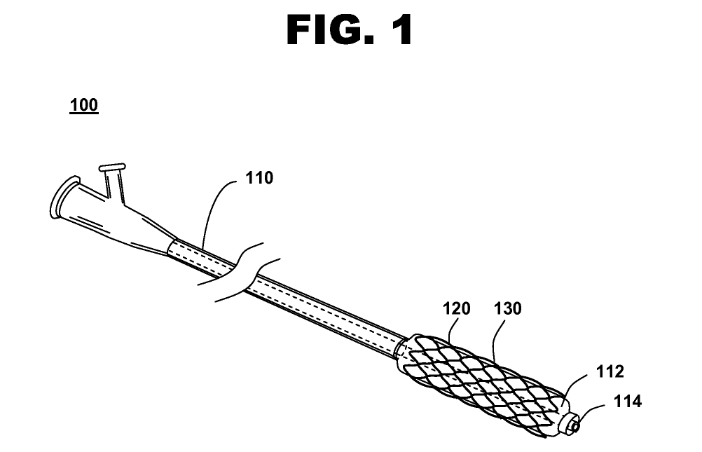

FIG. 1 shows an illustration of a system for treating a vascular condition, comprising a stent coupled to a catheter, in accordance with one embodiment of the present invention at 100. Stent with catheter 100 includes a stent 120 coupled to a delivery catheter 110. Stent 120 includes a stent framework 130 and, in some embodiments, a therapeutic agent 140 disposed on the stent framework 130. Therapeutic agent 140 includes at least a first therapeutic agent. In certain embodiments, therapeutic agent 140 includes at least two therapeutic agents—a first therapeutic agent and a second therapeutic agent, for example.

Insertion of stent 120 into a vessel in the body helps treat, for example, heart disease, various cardiovascular ailments, and other vascular conditions. Catheter-deployed stent 120 typically is used to treat one or more blockages, occlusions, stenoses, or diseased regions in the coronary artery, femoral artery, peripheral arteries, and other arteries in the body. Treatment of vascular conditions may include the prevention or correction of various ailments and deficiencies associated with the cardiovascular system, the cerebrovascular system, urinogenital systems, biliary conduits, abdominal passageways and other biological vessels within the body.

An exemplary therapeutic agent 140 includes or encapsulates one or more therapeutic agents. Therapeutic agent 140 may comprise one or more therapeutic agents dispersed within or encased by drug layers or barrier layers, such as an intermediate layer of magnesium, on stent 120, which are eluted or leached from stent 120 with, for example, controlled time delivery after deployment of stent 120 into the body. A therapeutic agent is capable of producing a beneficial effect against one or more conditions including coronary restenosis, cardiovascular restenosis, angiographic restenosis, arteriosclerosis, hyperplasia, and other diseases or conditions. For example, the therapeutic agent can be selected to inhibit or prevent vascular restenosis, a condition corresponding to a narrowing or constriction of the diameter of the bodily lumen where the stent is placed. Therapeutic agent 140 may comprise, for example, an antirestenotic agent such as rapamycin, a rapamycin derivative, or a rapamycin analog to prevent or reduce the recurrence of narrowing and blockage of the bodily vessel. Therapeutic agent 140 may comprise an anti-cancer drug such as camptothecin or other topoisomerase inhibitors, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or a combination thereof. In one example, a first therapeutic agent comprises an antirestenotic drug such as rapamycin, a rapamycin derivative, or a rapamycin analog. The second therapeutic agent may comprise, for example, an anti-cancer drug such as camptothecin or other topoisomerase inhibitors. The therapeutic agent constituency in the drug layers may be, for example, between 0.1 percent and 50 percent of the drug layer by weight. In another example, the first therapeutic agent comprises an anti-proliferative compound such as 5-fluorouracil, with an optional second therapeutic agent such as rapamycin, a rapamycin derivative, a rapamycin analog, or dexamethosone. In another example, the first therapeutic agent comprises an anti-inflammatant such as dexamethasone, and an optional second therapeutic agent such as 5-fluorouracil. In another embodiment, the therapeutic agent is one of a drug and a drug polymer.

In one example, a first therapeutic agent comprises an antirestenotic drug such as rapamycin, a rapamycin derivative, or a rapamycin analog. The second therapeutic agent may comprise, for example, an anti-cancer drug such as camptothecin or other topoisomerase inhibitors. The therapeutic agent constituency in the drug layers may be, for example, between 0.1 percent and 50 percent of the drug layer by weight. In another example, the first therapeutic agent comprises an anti-proliferative compound such as 5-fluorouracil, with an optional second therapeutic agent such as rapamycin, a rapamycin derivative, a rapamycin analog, or dexamethosone. In another example, the first therapeutic agent comprises an anti-inflammation agent such as dexamethasone, and an optional second therapeutic agent such as 5-fluorouracil.

The elution rates of the therapeutic agents and total drug eluted into the body and the tissue bed surrounding the stent framework are based on the target thickness of therapeutic agent 140, the constituency and individual layer thicknesses of therapeutic agent 140, the nature and concentration of the therapeutic agents, the thickness and composition of any cap coat, and other factors. Therapeutic agent 140 may include and elute or leach multiple therapeutic agents to achieve the desired therapeutic effect. In some cases, a portion of a topcoat layer is absorbed into the body.

Catheter 110 of an exemplary embodiment of the present invention includes a balloon 112 that expands and deploys the stent within a vessel of the body. After positioning stent 120 within the vessel with the assistance of a guide wire traversing through a guide wire lumen 114 inside catheter 110, balloon 112 is inflated by pressurizing a fluid such as a contrast fluid or saline solution that fills a tube inside catheter 110 and balloon 112. Stent 120 is expanded until a desired diameter is reached, and then the fluid is depressurized or pumped out, separating balloon 112 from stent 120 and leaving stent 120 deployed in the vessel of the body. Alternately, catheter 110 may include a sheath that retracts to allow expansion of a self-expanding version of stent 120.

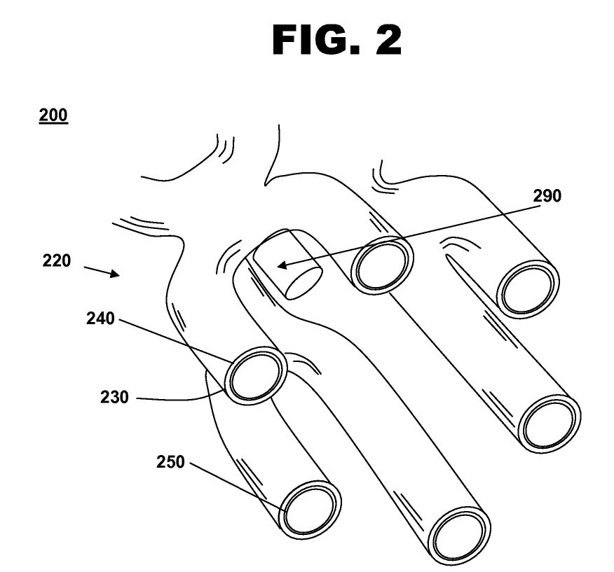

FIG. 2 shows a cross-sectional perspective view of a stent, in accordance with one embodiment of the present invention at 200. A stent 220 includes a stent framework 230 with a coating 240 disposed on stent framework 230. Coating 240 envelops and surrounds a layer of stem cells 250 disposed on the exterior surface of the stent framework. In addition, FIG. 2 illustrates electrical lead 290, disposed within the lumen defined by stent framework 230. In one embodiment, electrical lead 290 is connected directly to stent framework 230 and provides an electrical charge to the framework. In other embodiments, the electrical lead does not directly contact the framework, but is disposed within the lumen defined by the framework. In such embodiment, the electrical lead is carried within a lumen of the catheter. In yet other embodiments, the electrical lead is a device positioned outside the patient body and configured to induce an electrical field within the stent framework, such as by using a magnetic field and a metallic stent framework. Coating 240 can be polymeric, or non-polymeric. Additionally, in one embodiment, the layer of stem cells 250 includes nutrients, a growth medium or matrix, or other similar food to sustain the stem cells for a given span of time.

The stem cells incorporated within the stem cell layer are applied using an appropriate technique. For example, the stem cells can be inoculated on the stent framework using a growth medium, sprayed, dipped, or the like. In one embodiment, the stem cells are applied prior to shipping the stent to a medical facility, while in other embodiments, the stem cells are applied a short time, such as within one day, of the stent deployment. For example, the seeding density of stem cells can vary between 100 cells/cm² and 5000 cells/cm².

Although illustrated with one set of stem cell and coating layers, multiple sets of stem cell and coating layers may be disposed on stent framework 230. For example, ten sets of layers, each layer on the order of 0.1 micrometers thick, can be alternately disposed on stent framework 230 to produce a two-micrometer thick coating. In another example, twenty sets of layers, each layer on the order of 0.5 micrometers thick, can be alternately disposed on stent framework 230 to produce a twenty-micrometer thick coating. The stem cell layers and the coating layers need not be the same thickness, and the thickness of each may be varied throughout coating 240. Alternately, the first coating layer may be a barrier layer, and the final coating layer may comprise, for example, a thick cap coat.

Stent framework 230 comprises a metallic base or a polymeric base, such as stainless steel, nitinol, tantalum, MP35N alloy, platinum, titanium, a chromium-based alloy, a suitable biocompatible alloy, a suitable biocompatible material, a biocompatible polymer, or a combination thereof. The polymeric base material may comprise any suitable polymer for biomedical stent applications, as is known in the art.

In one example, coating layer 240 comprises a first polymer such as poly(ethylene-vinyl acetate) (PEVA) and a first therapeutic agent such as camptothecin, rapamycin, a rapamycin derivative, or a rapamycin analog. Coating layers 244, in certain embodiments, also comprise a second polymer such as polyurethane, polycaprolactone, or a blended polymer of polyurethane and polycaprolactone that can be selected based on a predetermined elution rate. For example, tailoring the fraction of the two polymers, the thickness of the drug-polymer layers and the barrier layers, or the concentration of the therapeutic agents controls the elution rate of one or more therapeutic agents dispersed within or encased by coating 240. Drug elution refers to the transfer of a therapeutic agent from coating 240 to the surrounding area in a body. The amount of drug eluted is determined as the total amount of therapeutic agent excreted out of coating 240, typically measured in units of weight such as micrograms, or in weight per peripheral area of the stent. In another embodiment, coating layers 240 includes a second therapeutic agent such as camptothecin. In another embodiment, the concentration of the therapeutic agents in either coating layer 240 is modulated to provide a predetermined drug-release profile. The concentration of the second therapeutic agent in coating layer 240 may be between, for example, 0.1 percent and 50 percent by weight. In addition, the type and concentration of stem cells in stem cell layer 250 can be modulated and differ along the span of the stent framework. For example, the crowns of each stent strut can be masked to reduce concentration of stem cells and coating on the crown regions that are exposed to relatively high levels of mechanical stress and/or strain.

In another example of a multi-layer coated stent, coating layer 240 comprises a first polymer including a rigid thermoplastic polyurethane and an anti-proliferative therapeutic agent such as 5-fluorouracil, or an ester-extended polyurethane. An example of a rigid thermoplastic polyurethane is TECOPLAST®, a hydrophobic polymer available from Thermedics Polymer Products in Wilmington, Mass. An example of an ester-extended polyurethane is TECOPHILIC®, a hydrophilic polymer also available from Thermedics Polymer Products in Wilmington, Mass. Coating layers 240 may optionally include a therapeutic agent such as rapamycin, a rapamycin derivative, or a rapamycin analog. Alternatively, coating layer 240 may include an anti-inflammatant such as dexamethasone.

In another example, coating layers 240 comprises a first polymer including a copolymer of methacrylamide, methacrylate, and vinyl alcohol with an anti-proliferative therapeutic agent such as 5-fluorouracil. In another example, coating layer 240 includes a second polymer such as rigid thermoplastic polyurethane and may include an anti-inflammatant such as dexamethasone. In another example, coating layers 240 comprise a first polymer including a copolymer of methacrylamide, methacrylate, and vinyl acetate with an anti-inflammatant such as dexamethasone, and/or a second polymer such as poly(butyl methacrylate) (PBMA), and may include a second therapeutic agent such as 5-fluorouracil.

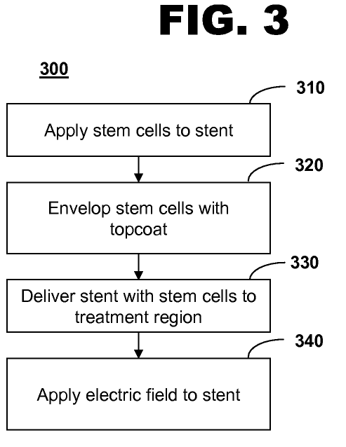

FIG. 3 illustrates one embodiment of a method 300 treating a vascular condition, in accordance with one aspect of the invention. Method 300 begins by applying, step 310, a plurality of stem cells to an exterior of a stent framework. In one embodiment, the exterior includes a plurality of surface features, such as surface modifications, to increase the carrying capacity of the stent framework. The applied stem cells are then enveloped, step 320, by a topcoat layer. The topcoat layer, in one embodiment, is polymeric. In another embodiment, the topcoat layer includes at least one drug-polymer. In yet another embodiment, the topcoat layer comprises a sacrificial element to leach from the stent framework on deployment, such as magnesium.

The coated stent is delivered, step 330, to a target region of a vessel. For example, the coated stent can be attached to a catheter, delivered to the target region and released from the catheter, such as with a balloon or via self-expansion. After delivery to the target region, an electrical field is applied, step 340, to the stent framework to induce angiogenesis of the stem cells. The electrical field can be directly induced via a lead attached to the stent framework, or indirectly induced via a magnetic field resulting from within the lumen of the stent, or from the exterior of the body of a patient. The field is applied for a predetermined span of time, such as 5-15 minutes, for induction of the angiogenesis. In one embodiment, the field assumes the approximate strength of the voltage differential generated by a skin wound, or approximately 1-420 Hz frequency, 30-120 V, 100 uA max. In another embodiment, the field is approximately 75-750 V/m held constant for 1-15 min. Other field strengths and time spans can be used, depending on the therapeutic goals, for example a series of treatments applying electrical stimulation across a span of several hours.

It is important to note that the figures herein illustrate specific applications and embodiments of the present invention, and are not intended to limit the scope of the present disclosure or claims to that which is presented therein. Upon reading the specification and reviewing the drawings hereof, it will become immediately obvious to those skilled in the art that many other embodiments of the present invention are possible, and that such embodiments are contemplated and fall within the scope of the presently claimed invention without departing from the spirit and scope of the invention. The scope of the invention is indicated in the appended claims, and all changes that come within the meaning and range of equivalents are intended to be embraced therein.

What is claimed is:

1. A method of treating a vascular condition, the method comprising:
applying a plurality of stem cells to an exterior surface of a stent;
enveloping the applied stem cells with a topcoat layer;
delivering the stent with applied stem cells and topcoat to a treatment region of a vessel within a body; and
applying an electrical field to the stent for a predetermined time.

2. The method of claim 1 further comprising:
attaching an electrical lead to the stent; and
applying an electrical field to the lead.

3. The method of claim 1 further comprising:
determining a magnetic region exterior the body so that a magnetic field produced by a magnet will induce the electrical field in the stent; and
placing the magnet in the magnetic region.

4. The method of claim 1 further comprising:
forming the topcoat layer.

5. The method of claim 3 wherein the topcoat layer comprises at least one of a drug and a drug polymer.

6. A system for treating a vascular condition, comprising:
a catheter;
a stent disposed on the catheter;
at least one layer of stem cells disposed on an exterior surface of the stent;
a topcoat layer surrounding the layer of stem cells; and
at least one electrical lead attached to the stent, the electrical lead operable to induce an electrical field around the stent.

7. The system of claim 5 wherein the topcoat comprises one of a drug and a drug polymer.

8. A system for treating a vascular condition, comprising:
a catheter;
a stent disposed on the catheter;
at least one layer of stem cells disposed on an exterior surface of the stent;
a topcoat layer surrounding the layer of stem cells; and
at least one magnetic device operable to induce an electrical field around the stent.

9. The system of claim 7 wherein the topcoat comprises one of a drug and a drug polymer.

10. The system of claim 7 wherein the magnetic device is disposed within the lumen defined by the stent.

11. The system of claim 7 wherein the magnetic device configured to induce the electrical field around the stent from a position exterior a patient body.
